(19) United States
(12) Patent Application Publication
(10) Pub. No.: US 2026/0283809 A1
(43) Pub. Date: Sep. 24, 2026

(54) ORTHOPAEDIC KNEE JOINT PROSTHESIS

(71) Applicant: Aesculap AG, Tuttlingen (DE)

(72) Inventors: Berna Richter, Muehlheim (DE); Rachel Lang, Chaumont (FR); Thomas Grupp, Denkingen (DE); Julius Dohm, Kronberg (DE)

(21) Appl. No.: 19/490,051

(22) PCT Filed: Jun. 6, 2024
(86) PCT No.: PCT/EP2024/065610

§ 371 Date: Dec. 4, 2025

(30) Foreign Application Priority Data
Jun. 6, 2023 (DE) 10 2023 114 759.5

(51) Int. Cl. A61F 2/38; A61F 2/30

(52) U.S. Cl. CPC A61F 2/3868; A61F 2/30767; A61F 2/3859; A61F 2/389; A61F 2002/30011; A61F 2002/30016; A61F 2002/30026; A61F 2002/30321; A61F 2002/3092; A61F 2002/3093; A61F 2310/00023; A61F 2310/00407; A61F 2310/00449; A61F 2310/00485; A61F 2310/00886; A61F 2310/00892

(57) ABSTRACT

An orthopedic knee joint prosthesis includes a metallic femoral implant part, a metallic tibial implant part, and a meniscus replacement part. The femoral implant part and in particular also the tibial implant part are manufactured additively by metallic 3D printing. The femoral implant part and/or the tibial implant part have/has a three-dimensionally porous surface structure formed by the 3D printing, in surface regions configured to face the bone, to allow incorporation of the bone into the implant part. The femoral implant part and/or the tibial implant part have/has at least in some areas a coating in surface regions configured to not be in contact with the bone and not be in contact with the meniscus replacement part but configured to be in contact with soft tissue. The coating reduces a surface roughness of the surface regions.

### CROSS-REFERENCE TO RELATED APPLICATIONS

This application is the United States national phase entry of International Application No. PCT/EP2024/065610, filed on Jun. 6, 2024, and claims priority to German Application No. 10 2023 114 759.5, filed on Jun. 6, 2023.

### FIELD

The invention relates to an orthopedic knee joint prosthesis having a metallic femoral implant part, having a metallic tibial implant part and having a meniscus replacement part which forms a sliding partner for the femoral implant part and is held on the tibial implant part, wherein the femoral implant part has surface regions facing the bone and in contact with the bone in the implanted state,
wherein the femoral implant part has articulating condylar surface regions in sliding contact with the meniscus replacement part as a sliding partner in the implanted state,
wherein the tibial implant part has surface regions facing the bone and in contact with the bone in the implanted state,
wherein the tibial implant part has surface regions in contact with the meniscus replacement part in the implanted state,
wherein the femoral implant part and the tibial implant part each have surface regions not in contact with the bone and not in contact with the meniscus replacement part but in contact with soft tissue in the implanted state,
wherein the femoral implant part has at least in some areas a hardening and wear-reducing, in particular multi-layer, coating on the articulating condylar surface regions in sliding contact with the sliding partner.

### BACKGROUND

Such a knee joint prosthesis is already known from EP 2 051 666 B1, which proposed forming a hardening and wear-reducing coating on the articulating condylar surface regions in the femoral implant part in sliding contact with the meniscus replacement part, and also on the surface regions of the tibial implant part in contact with the meniscus replacement part. This hardening and wear-reducing coating is multi-layer and has, in particular, ceramic layers, which prove to be particularly wear-resistant in sliding contact with corresponding articulation surfaces of the meniscus replacement part. They also prevent or reduce the release of metal ions from the interior of the femoral or tibial implant part as a result of the articulating sliding contact.

Since then, only attempts have been made to optimize the wear resistance of surface regions in the implant parts subject to stress through articulating sliding movements, including micromovements. A similar knee joint prosthesis is disclosed in U.S. Pat. No. 8,642,112 B2. Here, too, a hardening and wear-reducing coating is applied to articulating surface regions. To form a three-dimensionally porous surface structure facing and in contact with the bone, a separate porous layer is attached to the implant part. One embodiment involves a highly porous, open-cell biomaterial in the form of a reticulated carbon foam.

### SUMMARY

The object of the present invention is to achieve optimal anchoring of the metallic implant material in the bone, starting from a knee joint prosthesis of the type mentioned at the outset, and simultaneously to achieve unproblematic contact with the surrounding soft tissue.

This object is achieved according to the invention on the basis of a knee joint prosthesis of the type mentioned at the outset in that the femoral implant part and in particular also the tibial implant part are manufactured additively by metallic 3D printing, in that the femoral implant part and/or the tibial implant part have/has a three-dimensionally porous surface structure formed by metallic 3D printing, in surface regions facing the bone, to allow incorporation of the bone into the implant part, and in that the femoral implant part and/or the tibial implant part have/has at least in some areas a coating in surface regions not in contact with the bone and not in contact with the meniscus replacement part but in contact with soft tissue, by means of which coating a surface roughness of these surface regions is reduced.

On the one hand, it is proposed to provide a three-dimensionally porous surface structure in surface regions of the implant part in question facing the bone by metallic 3D printing. On the other hand, it is proposed to provide a coating that reduces the surface roughness of surface regions not in contact with the bone or the meniscus replacement part but in contact with soft tissue. It has been found, according to the invention, that rough surfaces in contact with soft tissue can lead to increased cell death during relative movement to soft tissue. The present invention therefore proposes to specifically reduce the surface roughness of these surface regions of the implant parts in contact with soft tissue by applying a coating, so that irritation of the surrounding soft tissue is reduced. The surface roughness is lower than that of the uncoated surface regions of the 3D-printed implant part in question, and also lower than that of the surface regions of the implant part in question facing the bone. As a result, an optimization of preferably all of the above-mentioned surface regions of the implant part in question is achieved with regard to their corresponding specific function in the implanted state of the prosthesis. For this purpose, the implant parts are advantageously manufactured in an additive manufacturing process using metallic 3D printing, wherein any surface roughness created in this process can be smoothed by applying the coating. This coating in surface regions in contact with soft tissue reduces or even prevents the tendency for bone to incorporate into the implant part, in particular in regions near the joint and at the transition to resection surfaces; thus, undesirable heterotopic ossification can be prevented or reduced. This proves particularly advantageous in conjunction with implant parts made of titanium or titanium alloy, as bone incorporates particularly well into titanium.

The coating can advantageously prevent or reduce bacterial biofilm formation through the use of suitable materials, such as zirconium nitride (ZrN). Such biofilm formation is associated, for example, with infections that require revision surgery.

It may be found that the surface roughness of surface regions not in contact with the bone and not in contact with the meniscus replacement part but in contact with soft tissue and provided with the coating is greater than or equal to a surface roughness of the articulating condylar surface regions in sliding contact and provided with the hardening and wear-reducing coating. Advantageously, surface regions in contact with soft tissue in the femoral implant part and/or the tibial implant part are designed to be as smooth as possible.

It proves to be advantageous if the surface roughness of surface regions not in contact with the bone and not in contact with the meniscus replacement part but in contact with soft tissue and provided with the coating is at most 0.50 μm, in particular at most 0.40 μm, in particular at most 0.30 μm and in particular at least 0.04 μm, in particular at least 0.05 μm and in particular at least 0.10 μm, measured as Ra according to DIN/ISO 21920-3.

Various methods can be used to produce the coating that reduces surface roughness. It proves to be advantageous if this coating in surface regions not in contact with the bone and not in contact with the meniscus replacement part but in contact with soft tissue is produced by PVD (Physical Vapor Deposition) processes. This allows for a very uniform and thin coating to be produced in a process-stable manner. It is particularly advantageous if the coating has a thickness of 3.5-6.0 μm, which can be measured and determined according to DIN/ISO 26423. Ceramic materials, such as in particular zirconium nitride (ZrN), chromium nitride (CrN) or chromium carbonitride (CrCN) are suitable for the coating, which can preferably be multi-layer. The coating can be bonded to the implant part via an adhesion-promoting layer, in particular one based on cobalt-chromium or titanium.

In an advantageous embodiment of the knee joint prosthesis according to the invention, it is provided that at least the tibial implant part has a three-dimensionally porous surface structure in surface regions facing the bone, to allow incorporation of the bone into the implant part, and that the tibial implant part has at least in some areas a coating in surface regions not in contact with the bone and not in contact with the meniscus replacement part but in contact with soft tissue, by means of which coating a surface roughness of these surface regions is reduced. This allows for secure anchoring of the tibial implant part in the bone, while also reducing irritation to the surrounding soft tissue in the region of the tibial implant part as a result of the smooth surface in contact with soft tissue.

The hardening and wear-reducing coating in the articulating surface regions in sliding contact with the meniscus replacement part is described below. It proves to be advantageous if the hardening and wear-reducing, in particular multi-layer, coating in the articulating condylar surface regions in sliding contact comprises a ceramic surface, in particular made of or based on zirconium nitride (ZrN).

Furthermore, it proves to be advantageous if the hardening and wear-reducing coating in the articulating condylar surface regions in sliding contact is formed in multiple layers and is bonded to the implant part via an adhesion-promoting layer, in particular based on titanium.

Furthermore, it proves to be advantageous if the hardening and wear-reducing coating has layers based on chromium nitride (CrN) and/or based on chromium carbonitride (CrCN) and/or based on zirconium nitride (ZrN).

Furthermore, it proves to be advantageous if the hardening and wear-reducing coating has a surface layer based on zirconium nitride (ZrN) and inner layers based on chromium nitride (CrN) or chromium carbonitride (CrCN), wherein multiple inner layers based on chromium nitride (CrN) and chromium carbonitride (CrCN) can alternate.

It is also advantageous if not only the articulating condylar surface regions of the femoral implant part, but also the tibial implant part has a hardening and wear-reducing, in particular multi-layer, coating of the type just described in surface regions in contact with the meniscus replacement part in order to reduce wear caused by micromovements between the meniscus replacement part and a plateau of the tibial implant part.

In order to bond the surface roughness-reducing coating in the soft tissue contact region, it is advantageous if the coating in surface regions not in contact with the bone and not in contact with the meniscus replacement part but in contact with soft tissue is bonded to the implant part via a titanium-based adhesion-promoting layer.

It has been found according to the invention that it is possible and even advantageous if the coating in surface regions not in contact with the bone and not in contact with the meniscus replacement part but in contact with soft tissue is the same coating as the hardening and wear-reducing coating in the articulating condylar surface regions in sliding contact. It is conceivable that the coatings are bonded to a base material of the corresponding implant part via various adhesion-promoting layers. For example, it would be conceivable that the adhesion-promoting layer in the hardening and wear-reducing coating is based on cobalt-chromium or titanium, and that the adhesion-promoting layer in the surface roughness-reducing coating in the soft tissue contact region is based on titanium. However, both coatings can also be bonded via the same titanium-based adhesion-promoting layer.

It is furthermore advantageous if the meniscus replacement part has a polymer-based, in particular polyethylene-based, sliding surface in contact with the articulating condylar surface regions of the femoral implant part. The meniscus replacement part can also be made entirely of polymer.

With regard to the production of the implant parts, it proves to be advantageous that the metallic femoral implant part and the metallic tibial implant part are manufactured additively.

Additive manufacturing in the form of metallic 3D printing is suitable for the production of complex and in particular delicate structures that would be difficult or impossible to produce using injection molding or casting technology. With regard to the present invention, however, additive manufacturing proves to be advantageous in that it can be used to produce any closed and/or three-dimensionally porous structures, including surface structures. For example, an implant part can be designed with both a preferably substantially closed articulating surface region and a three-dimensionally porous surface region in contact with the bone. The three-dimensionally porous surface region on the corresponding implant part can thus be produced by additive manufacturing in the form of metallic 3D printing, without the need to add an additional porous layer to the implant part. In particular, it is conceivable that a corresponding implant part can be formed with a three-dimensional bar structure in the region of bone contact, wherein the three-dimensional bar structure can extend inward to any desired depth starting from the outer shell surface. This allows the bone tissue to penetrate deeply into the implant part, allowing for a lasting, secure fit of the implant part and thus of the prosthesis. A certain surface roughness inherent in 3D printing can then be reduced in the soft tissue contact region by applying the roughness-reducing coating.

It proves to be particularly advantageous if the metallic femoral implant part and/or the metallic tibial implant part are/is additively manufactured from titanium or titanium alloy.

Titanium or titanium alloy is ideally suited for the direct formation of surface regions facing and in contact with the bone, as bone incorporates well into surfaces formed by titanium or titanium alloy. The natural surface roughness created by 3D printing proves to be additionally beneficial for this purpose. In particular, an alpha or beta titanium alloy, for example Ti6-A14-V or Ti-24Nb-4Zr-8Sn, can be used as the titanium alloy.

By applying the previously formed coatings to articulating condylar surface regions, the tribological properties of titanium surfaces can be improved, and in the soft tissue contact region, undesirable bone incorporation can be prevented or at least inhibited, as already mentioned at the outset.

The subject matter of the invention is also considered to be a corresponding implant part, i.e., a femoral implant part and/or a tibial implant part, which have/has a three-dimensionally porous surface structure in the region of bone contact in surface regions facing the bone, to allow incorporation of the bone into the implant part, and which have/has at least in some areas a coating in surface regions not in contact with the bone and not in contact with the meniscus replacement part but in contact with soft tissue, by means of which coating a surface roughness of these surface regions is reduced.

### BRIEF DESCRIPTION OF THE DRAWINGS

Further features, details and advantages of the invention can be found in the drawings and in the following description of an embodiment according to the invention of the orthopedic knee joint prosthesis.

### In the Drawings:

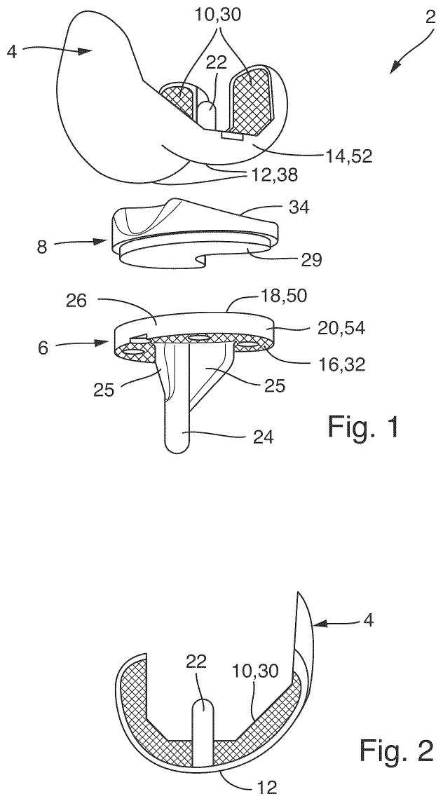

FIG. 1 is an exploded view of an embodiment according to the invention of an orthopedic knee joint prosthesis having a femoral implant part, a tibial implant part and a meniscus replacement part fixable to the tibial implant part;

FIG. 2 is a schematic, simplified sectional view of the femoral implant part;

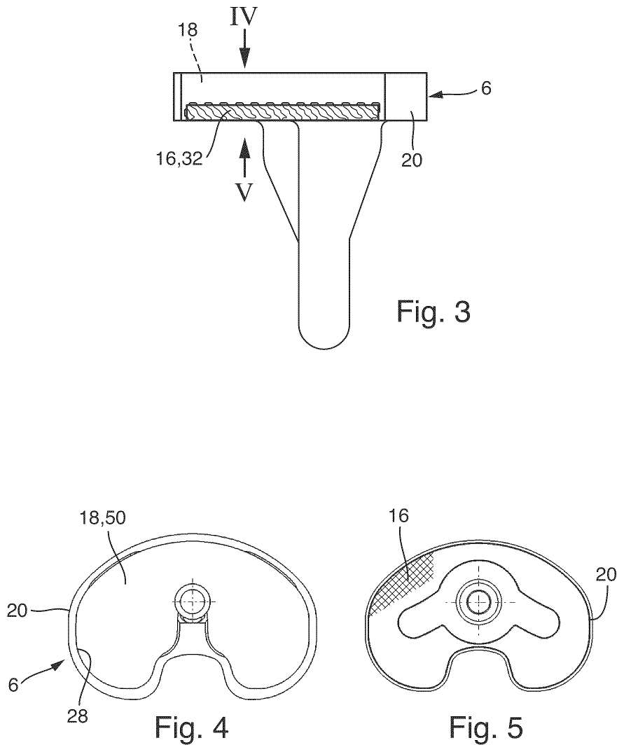

FIG. 3 is a side view of the tibial implant part with partial cross section;

FIG. 4 is a view of the tibial implant part from above in the direction of arrow IV in FIG. 3;

FIG. 5 is a view from below in the direction of arrow V in FIG. 3;

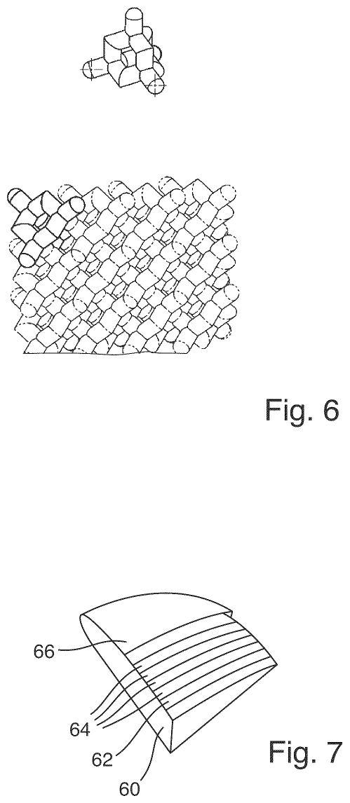

FIG. 6 schematically shows an additively manufactured, three-dimensionally porous metallic structure of an implant part region for bone tissue incorporation; and

FIG. 7 schematically illustrates a multi-layer coating.

### DETAILED DESCRIPTION

FIG. 1 shows, in an exploded view, an orthopedic knee joint prosthesis, denoted as a whole by reference sign 2, having a femoral implant part 4, a tibial implant part 6 and a meniscus replacement part 8, which can typically be anchored to the tibial implant part 6.

The femoral implant part 4 has surface regions 10 facing a bone of the thigh (femur) (not shown) and in contact with the bone, and furthermore has articulating condylar surface regions 12 in sliding contact with the meniscus replacement part 8 as a sliding partner. Furthermore, the femoral implant part 4 has surface regions 14 not in contact with the bone and not in contact with the meniscus replacement part 8 but in contact with soft tissue. These are surface regions 14 located outside the articulating sliding contact with the meniscus replacement part, in particular those horizontally flanking the femoral implant part 4.

The tibial implant part 6 also has surface regions 16 facing the bone of the lower leg (tibia) and in contact with the bone. Furthermore, the tibial implant part 6 has surface regions 18 in contact with the meniscus replacement part 8, and finally, the tibial implant part 6 also has surface regions 20 not in contact with the bone and not in contact with the meniscus replacement part 8 but in contact with soft tissue.

It can be seen from FIG. 1 to 5 that the femoral implant part 4 has at least one dome-shaped or shaft-shaped projection 22, and the tibial implant part 6 has an elongate projection 24, which each serve for engagement and anchoring on the thigh or lower leg bone, respectively. The projection 24 of the tibial implant part 6 is also attached to a disc-shaped plateau part 26 of the tibial implant part 6, via cheek-like portions 25 extending in the vertical plane.

As best seen from FIG. 4, the disc-shaped plateau part 26 has a flat recess 28 facing away from the bone and toward the meniscus replacement part 8 for inserting and, in particular, locking the complementary region 28 of the meniscus replacement part 8.

In the preferred case shown by way of example, the femoral implant part 4 and the tibial implant part 6 are each designed having a three-dimensionally porous surface structure 30 or 32, respectively, in the region of their bone contact. In both cases, this three-dimensionally porous surface structure 30 and 32 extends macroscopically over at least 0.5 mm and in particular up to 20 mm, in particular up to 15 mm and in particular up to 10 mm in the depth direction into the corresponding implant part 4 or 6, respectively, which are each preferably produced in additive manufacturing processes.

The meniscus replacement part 8 forms, as a sliding partner to the femoral implant part 4, three-dimensionally shaped sliding surface regions 34 which are substantially complementary in shape to the condylar surface regions 12 of the femoral implant part 4 and which, like in particular the entire meniscus replacement part 8, are formed from a polymer, in particular polyethylene. In order to reduce frictional contact and thus wear between the condylar surface regions 12 of the femoral implant part 4 and the meniscus replacement part 8, the articulating condylar surface regions 12 in sliding contact with the meniscus replacement part 8 have a hardening and wear-reducing, in particular multi-layer, coating 38. This coating has multiple ceramic layers as well as a surface layer, for example and preferably based on zirconium nitride, as well as an adhesion-promoting layer on the base material of the femoral implant part 4 (described below by way of example in connection with FIG. 7). The hardening and wear-reducing coating 38 can be formed in detail as described in EP 2 051 666 B1, so that the relevant content of this document with regard to the structure of the hardening and wear-reducing coating is incorporated by reference into the present application.

A corresponding hardening and wear-reducing coating 50 is present on the surface region 18 of the tibial implant part 6 in contact with the meniscus replacement part 8. This is advantageous because, despite the retaining connection, in particular the locking connection, between the meniscus replacement part 8 and the tibial implant part 6, micromovements occur between these parts.

With the present invention, it was discovered that surface regions of the femoral implant part 4 or the tibial implant part 6, respectively, in contact with soft tissue can have a problematic effect on the soft tissue and, in the worst case, can lead to cell death if these surface regions 14 or 20, respectively, are too rough and, as a result of the articulating movement of the implant parts against one another, a relative movement occurs between these surface regions and the soft tissue. Therefore, the present invention proposes providing surface regions 14 of the femoral implant part 4 in contact with soft tissue and surface regions 20 of the tibial implant part 6 outside the contact region with the bone and the meniscus replacement part with a smoothing coating 52 or 54, respectively, by means of which the surface roughness of these surface regions 14, 20 in contact with soft tissue is reduced. The aim is to achieve a surface that is as smooth as possible so that relative movements do not lead to, or at least significantly reduce, the introduction of frictional energy into the soft tissue, which could prove problematic.

It has also proven to be advantageous that this surface roughness-reducing coating 52 or 54, respectively, is deposited only on the outer webs, struts or ribs of the structure during application in the PVD process in the event of an inadvertent coating of three-dimensionally porous surface regions 10 or 16, respectively, of the implant parts 4, 6 in contact with the bone, which does not noticeably impede the anchoring of bone material in the three-dimensionally porous structure.

It has furthermore been determined that the same layer structure can be used to form the smoothing, i.e., surface roughness-reducing coating 52, 54 as is used to produce the hardening and wear-reducing coating 38 or 50, respectively, in the articulating condylar surface regions 12 of the femoral implant part 4 and the surface regions 18 of the tibial implant part 6 facing the meniscus replacement part 8.

FIG. 7 schematically shows, by way of example, a multi-layer coating structure, which is equally suitable for the construction of the corresponding hardening and wear-reducing coating 38, 50 and for the smoothing coating 52, 54. A base material 60 of the corresponding implant part 4 or 6 and an adhesion-promoting layer 62, in particular a titanium-based layer, which is in particular only 30 to 200×10⁻⁹ m thick, can be seen. Multiple ceramic layers 64, in particular made of two alternating materials, are provided on top of this layer. Finally, a ceramic surface layer 66, in particular based on zirconium nitride, is provided. As mentioned, this layer structure can be used both as a hardening and wear-reducing coating 38, 50 and as a smoothing coating 52, 54 exclusively in contact with soft tissue.

1.-14. (canceled)

15. An orthopedic knee joint prosthesis comprising:
a femoral implant part;
a tibial implant part; and
a meniscus replacement part,
the femoral implant part and the tibial implant part being metallic,
the meniscus replacement part forming a sliding partner for the femoral implant part and being held on the tibial implant part,
the femoral implant part comprising first surface regions configured to face bone and be in contact with bone in an implanted state,
the femoral implant part also comprising articulating condylar surface regions configured to be in sliding contact with the meniscus replacement part as a sliding partner when in the implanted state,
the tibial implant part comprising second surface regions configured to face bone and be in contact with bone when in the implanted state,
the tibial implant part also comprising third surface regions configured to be in contact with the meniscus replacement part when in the implanted state,
the femoral implant part and the tibial implant part each further comprising fourth surface regions configured to be out of contact with bone, out of contact with the meniscus replacement part, and in contact with soft tissue when in the implanted state,
the femoral implant part comprising a first coating at least in some areas, the first coating being a hardening and wear-reducing coating in the articulating condylar surface regions in sliding contact with the sliding partner,
at least one of the femoral implant part and the tibial implant part comprising a three-dimensionally porous surface structure in surface regions that face bone when in the implanted state,
the three-dimensionally porous surface structure configured to allow incorporation of bone into the three-dimensionally porous surface structure, and
at least one of the femoral implant part and the tibial implant part further comprising at least in some areas a second coating in the fourth surface regions, the second coating configured to reduce a surface roughness of the fourth surface regions.

16. The orthopedic knee joint prosthesis according to claim 15, wherein the surface roughness of the fourth surface regions is greater than or equal to a surface roughness of the articulating condylar surface regions.

17. The orthopedic knee joint prosthesis according to claim 15, wherein the surface roughness of the fourth surface regions is at most 0.50 μm, measured as Ra according to DIN/ISO 21920-3.

18. The orthopedic knee joint prosthesis according to claim 15, wherein:
the tibial implant part has the three-dimensionally porous surface structure, and
the tibial implant part has at least in some areas the second coating in the fourth surface regions.

19. The orthopedic knee joint prosthesis according to claim 15, wherein the first coating comprises a ceramic surface.

20. The orthopedic knee joint prosthesis according to claim 19, wherein the ceramic surface comprises zirconium nitride.

21. The orthopedic knee joint prosthesis according to claim 15, wherein the first coating is formed in multiple layers and bonded to the femoral implant part via an adhesion-promoting layer.

22. The orthopedic knee joint prosthesis according to claim 21, wherein the adhesion-promoting layer comprises chromium or titanium.

23. The orthopedic knee joint prosthesis according to claim 15, wherein the first coating has layers comprising at least one of chromium nitride, chromium carbonitride, or zirconium nitride.

24. The orthopedic knee joint prosthesis according to claim 15, wherein the first coating has a surface layer comprising zirconium nitride and inner layers comprising chromium nitride or chromium carbonitride.

25. The orthopedic knee joint prosthesis according to claim 24, wherein the inner layers comprise alternating layers of chromium nitride and chromium carbonitride.

26. The orthopedic knee joint prosthesis according to claim 15, wherein the tibial implant part has a third coating in surface regions configured to be in contact with the meniscus replacement part in order to reduce wear caused by micromovements between the meniscus replacement part and a plateau of the tibial implant part.

27. The orthopedic knee joint prosthesis according to claim 26, wherein the third coating comprises a multi-layer coating.

28. The orthopedic knee joint prosthesis according to claim 15, wherein the second coating is bonded to the femoral implant part and/or tibial implant part via a titanium-based adhesion-promoting layer.

29. The orthopedic knee joint prosthesis according to claim 15, wherein the first coating and the second coating are the same.

30. The orthopedic knee joint prosthesis according to claim 15, wherein the meniscus replacement part has a polymer-based sliding surface in contact with the articulating condylar surface regions of the femoral implant part.

31. The orthopedic knee joint prosthesis according to claim 30, wherein the polymer-based sliding surface is polyethylene-based.

32. The orthopedic knee joint prosthesis according to claim 15, wherein the femoral implant part and/or the tibial implant part comprise titanium or titanium alloy.

33. The orthopedic knee joint prosthesis according to claim 15, wherein the first coating comprises a multi-layer coating.
